(19) United States
(12) Patent Application Publication
(10) Pub. No.: US 2023/0392265 A1
(43) Pub. Date: Dec. 7, 2023

(54) INTEGRATED GAS GENERATOR AND ELECTRICITY STORAGE SYSTEM

(71) Applicant: Wolfgang WINKLER, Winsen (DE)

(72) Inventor: Wolfgang WINKLER, Winsen (DE)

(21) Appl. No.: 18/032,082

(22) PCT Filed: Oct. 6, 2021
(86) PCT No.: PCT/EP2021/077520

§ 371 Date: Apr. 14, 2023

(30) Foreign Application Priority Data
Oct. 15, 2020 (DE) 10 2020 006 326.8

(51) Int. Cl. C25B 1/042; C25B 15/021; H01M 16/00; C07C 1/12; C07C 1/04

(52) U.S. Cl. CPC C25B 1/042; C25B 15/021; H01M 16/003; C07C 1/12; C07C 1/041

(57) ABSTRACT

A modular reactor configuration for the production of hydrogen (H₂) by means of electrolysis in its single-stage design and of methane (CH₄) in its two-stage design with optional gas storage and gas utilization in fuel cells, wherein the single-stage design, consisting of the electrolyzer, the fuel cell, the gas storage tanks for separate storage of H₂ and oxygen (O₂), the associated lines, the condenser, the H₂O container, the heat storage tanks and the evaporator, is based on the principles of a reversible product cycle for H₂ according to FIG. 1 and can serve both as electricity storage and for H₂ production as fuel gas, and whose two-stage design, exemplified according to FIG. 5 with the additional components the methanation reactor, the lines and, the heat exchangers and as well as the CH₄ discharge in the H₂O condenser, based on extended reversible reference processes, which describe the possible methanation reactions in this second reactor stage with the reaction equations, which can also run in parallel, and are thermodynamically equivalent to the reverse reaction of the oxidation of CH₄ and thus indicate the best possible structures for further technical implementation.

Without reliable storage technology, neither the political goals of the German government nor the EU's “Green Deal” will be able to be implemented in practice. Throughout living nature, the three elements C, H and O are the basis of energy storage. Their present reaction products water (H₂O) and carbon dioxide (CO₂) are ideally sufficient to produce synthetic fuels such as hydrogen (H₂), as well as hydrocarbons (CₙHₘ) especially methane (CH₄) and ethene (C₂H₄), generally hydrocarbon compounds, from fluctuating regenerative electricity with the help of “power-to-gas” technologies and to use them as regenerative storage substance, but also as sustainable industrial raw material. For this purpose, however, it is imperative to increase energy efficiency in the technical implementation. As described in (M. Thema, F. Bauer, M. Sterner: Power-to-gas: electrolysis and methanation status review. Renewable and Sustainable Energy Reviews 112 (2019) p 775-787 Table 2), the average efficiency for H₂ electrolysis is 77% and 41% for methanation of H₂ with CO or CO₂, which is essentially based on the Sabatier reaction, although lower values are certainly cited in the literature. Since H₂ is an essential feedstock for the implementation of the Sabatier reaction, all the processes mentioned above are also linked to the efficient supply of H₂.

The task of the invention is to significantly improve the above-mentioned efficiencies for “power-to-gas” technologies with the aid of an integrated overall concept, to minimize investment costs by means of new circuits and their constructive design, and thus to contribute to sustainable system integration that guarantees a secure and stable power supply despite fluctuating feed-in and contributes to a sustainable circular economy through CO₂ recycling.

### Problem Analysis, Solution Approaches and Task Definition

A problem analysis carried out with the help of the methodology of reversible process structures described in (Wolfgang Georg Winkler: Sustainable product development based on second law of thermodynamics. September 2011. Applied Energy 88(9): 3248-3256 DOI: 10.1016/j.apenergy.2011.03.020), showed that mainly two effects with their impact on further process integration lead to these relatively low efficiencies. These are the heat requirement for the evaporation of the supplied liquid H₂O and the separation of the O₂ produced during the thermal methanation by an oxidation with H₂ to be additionally produced for this purpose and the subsequent condensation of the H₂O produced. In addition, there are various deficiencies in system integration that are easily avoidable with the reversible structure as a design basis.

In electrolytic H₂ generation, H₂O is supplied in a liquid state and the necessary evaporation heat is generated electrically. This reduces the theoretically possible efficiency of H₂O electrolysis to 83% (Wolfgang Winkler, Ai Suzuki, Akira Miyamoto, Harumi Yokokawa, Mark C. Williams: Performance Envelope for Electrolyser Systems. ECS Trans. 2015 65(1): 253-262). From the consideration of reversible process control of electricity storage using H₂ according to (Wolfgang Georg Winkler: Sustainable product development based on second law of thermodynamics. September 2011. Applied Energy 88(9): 3248-3256 DOI: the necessary improvement options can be derived. For this purpose, the starting point for the overall electrolysis processes considered here are the reaction equations:

H₂O=H₂½O₂ (1)

and

CO₂=CO+½O₂. (2)

Whereby the last equation is only important for the integration of the electrolysis processes for CH₄ production. Both processes are thermodynamically similar, however, Eq. (2) for the representation of carbon monoxide (CO) is relevant here only for processes in the gas phase and thus, initially, for the analysis of the optimal system integration of the evaporation process, only the reversible process control based on Eq. (1) is relevant.

Electrolysis processes today operate at either high or low temperatures, so the thermodynamic differences between these two process options need to be explained. The reason for the additional power supply, to cover the heat requirement for evaporation in electrolysis is that H₂O is required for the reaction in gaseous form and not in liquid form as it is supplied. Recovery of the heat of condensation of the combustion exhaust gases given off to the environment is impracticable, and the issue is generally addressed formally in technical publications by the “thermoneutral voltage” defined with the higher heating value (compare, for example, “Electrochemical Thermodynamics,” Karl Winnacker Institute DECHEMA, https://dechema.de/kwi_media/Downloads/ec/5++Elektrochemisch+Thermodynamic s-p-976.pdf), which thus simply includes the evaporation heat in the voltage calculation. Not discussed is the possibility of using a heat pump to thermodynamically upgrade low-value heat with it and then use it for evaporation, thus reducing power requirements.

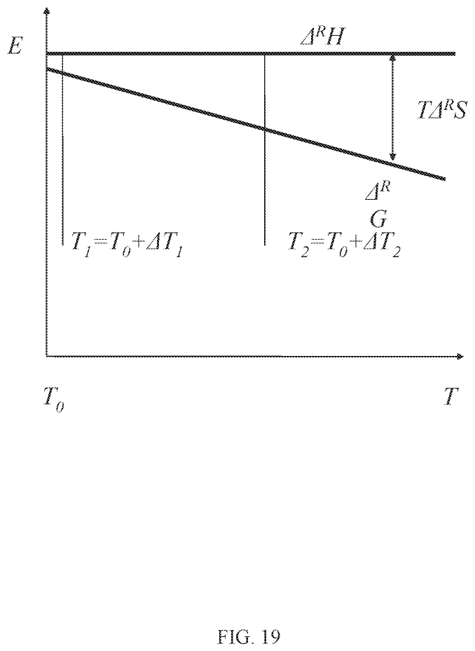

Also significant for the thermal system design is the temperature at which the electrolyzer must be operated in order to convert as much excess renewable electricity as possible into the chemical potential of the H₂ when used as an electricity storage system. To explain this is the task of FIG. 19, where the temperature-dependent distribution of the reversible work Δ^{R}G and reversible heat T·Δ^{R}S to be supplied during electrolysis is plotted above the temperature T, starting with the ambient temperature T₀, in order to obtain the reaction enthalpy Δ^{R}H required for the reaction. It follows directly from FIG. 19 that the higher the temperature T, the less reversible work Δ^{R}G can be converted to chemical potential. Thus, if the reversible work is available as fluctuating electric work, less electric work can be stored by means of a high-temperature electrolysis, operated at temperature T₂, for the same hydrogen production than by a low-temperature electrolysis, operated at temperature T₁. The heat requirement for evaporation, assuming the same pressure, depends only on the amount of H₂ produced. Therefore, a high temperature electrolysis requires more heat of evaporation, relative to the electrical work supplied, than a low temperature electrolysis. This shows that low-temperature electrolysis best fulfills the task of converting excess electricity and non-usable waste heat into usable chemical potential.

In contrast, high-temperature electrolysis is the more suitable process when sufficient high-temperature heat is available and little electrical power is available. With sufficient external heat supply, the required evaporation heat can also be provided without difficulty and without generating additional electricity consumption. However, it must be taken into account that the use of high-temperature heat for evaporation leads just as much to considerable exergy losses and thus does not solve the thermodynamic problem. The methanation processes in use today use H₂ and CO₂ as reactants. Optimization of electrolysis is therefore a basic prerequisite for further optimization of these processes.

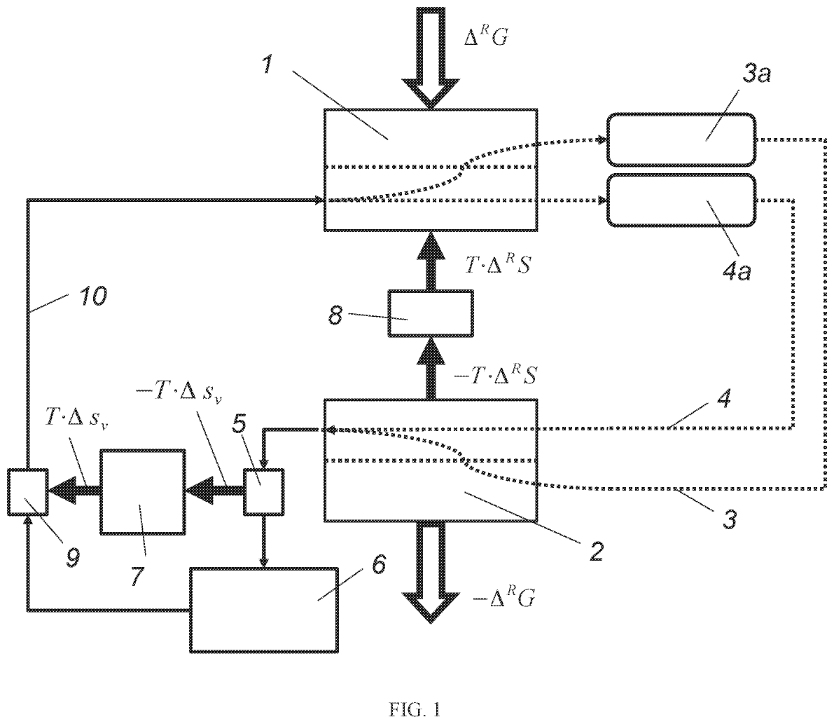

FIG. 1 shows the process flow diagram of a reversible H₂ electricity storage process, in which the thermodynamic variables required for balancing are entered for easier orientation. With this approach, the entire product cycle of the H₂ is considered from the extraction of the H₂O in the liquid state via its evaporation, the extraction of the gaseous products H₂ and O₂ by means of electrolysis with the supplied electrical work, the storage of the products, the subsequent conversion in the fuel cell with the output of the generated electrical work and condensation of the gaseous reaction product H₂O up to the discharge of the liquid H₂O. Since reversibility of all processes is assumed, occurring losses can only be caused by faulty system structures, which are thus easily identifiable. It is then the task of the technical implementation to come as close as possible to these theoretical structures with technically and economically reasonable solutions. However, their influence on system efficiency always remains directly identifiable by comparison with the ideal solution.

The main components of this isothermal system are the electrolyzer (1) and the fuel cell (2), which are interconnected via the two gas reservoirs (3a) and (4a) and the associated lines (3) and (4) and are operated at temperatures T above the associated saturated steam temperature, practically above 100° C. The conduit system (3) contains H₂ in the case of an H⁺-conducting electrolyte and O₂ in the case of an O²⁻-conducting electrolyte, and accordingly the conduit system (4) is filled with O₂ in the case of H⁺-conducting electrolytes and with H₂ in the case of O²⁻-conducting electrolytes. However, this is irrelevant for thermodynamic considerations as long as H₂ and O₂ remain separate in systems 3 and 4. The gases H₂ and O₂ stored separately in the gas storage tanks (3a) and (4a) are fed to the fuel cell (2) when required (lack of current) and are converted back to H₂O there, and the free enthalpy of reaction −Δ^{R}G is released to the outside as reversible work (here and in the following, the resulting signs are prefixed for better understanding and the quantities Δ^{R}G, Δ^{R}S and Δsᵥ are then to be understood consistently as absolute values). H₂O is condensed in the condenser (5) and fed to the H₂O tank (6) and the condensation heat −T·Δsᵥ is transferred to the heat accumulator (7), where Δsᵥ stands for the entropy change due to the phase change. In parallel, the reversible waste heat −T·Δ^{R}S generated by the fuel cell (2) due to the reaction entropy Δ^{R}S must be supplied to the heat accumulator (8). The liquid H₂O taken from the H₂O tank (6) in case of excess of current is fed to the evaporator (9), where it is evaporated by means of the supply of the required heat of evaporation +T·Δsᵥ from the heat accumulator (7) and fed to the electrolyzer (1) via the H₂O line (10). The electrolyzer (1) is supplied with the free enthalpy of reaction +Δ^{R}G as reversible work from outside and the heat supply +T·Δ^{R}S required because of the reaction entropy Δ^{R}S is supplied from the heat accumulator (8). This closes the circuit and describes the functioning of its components. If we now summarize the energy supplied to and removed from the system, the following applies to the supplied energy E_{zu}:

E_{zu}=Δ^{R}G+T·Δ^{R}S+T·Δsᵥ (3)

and for the energy to be removed E_{ab}:

E_{ab}=−Δ^{R}G−T·Δ^{R}S−T·Δsᵥ (4)

This process structure is exactly loss-free and real occurring losses in such plants are only due to the imperfection of the practical implementation with real lossy components. If we consider only the production of hydrogen which is spent elsewhere, the possibilities of recuperation via heat accumulators are omitted and even with fully reversible components, the heats T·Δ^{R}S and T·Δsᵥ have to be supplied from outside. It follows immediately that the main loss of H₂ production is that H₂O is supplied in the liquid state in most electrolyzers in use, and no heat stores exist to allow recuperation of heat given off elsewhere during oxidation of the H₂ at other times. However, the contribution of reaction entropy to heat demand is comparatively small in this case.

An important approach of the “power-to-gas” technology, however, refers precisely to the use of the synthetic gases at a spatial distance from the place where they are produced, which means that the direct spatial connection between fuel cell and electrolysis no longer exists and thus the internal heat exchange necessary for high efficiency is no longer possible. In theory, however, this dilemma can initially be remedied by overcoming the spatial separation of the connection between reversible electrolysis and reversible fuel cell by shifting the system border of the storage process as shown in FIG. 1 in such a way that the environment simultaneously becomes a heat sink for the fuel cell and a heat source for the electrolyzer. This succeeds according to (Wolfgang Georg Winkler: Sustainable product development based on second law of thermodynamics. September 2011. applied energy 88(9): 3248-3256 DOI: 10.1016/j.apenergy.2011.03.020) using a reversible heat-power process that reversibly cools the H₂O exiting the fuel cell at the condenser (5) to the ambient state now prevailing in the H₂O tank (6), and using a reversible heat pump that preheats the liquid H₂O of the H₂O tank (6) needed to operate the electrolyzer and evaporates it in the evaporator (9). Since the work generated by the reversible heat engine, is equal to the work required by the reversible heat pump, the reversibility of the overall system is maintained. However, this initially purely theoretical consideration provides some guidance as to how, in the case of isolated synthetic gas generation without the inclusion of the gas consumers themselves, an improvement in efficiency can be achieved with the aid of reducing the losses to provide the evaporation heat. Practicable ways to exploit this principle include using solar heat or geothermal sources to vaporize the H₂O needed for electrolysis, or waste heat from any process, reheated by heat pumps if necessary. This is the first step towards improving the efficiency of the generation of H₂ and thus of CH₄, as well as for the “power-to-gas” technology as a whole.

This will be explained in more detail in the following. As mentioned above, electrolyzer and fuel cell are usually operated separately, which means that the direct spatial connection between fuel cell and electrolysis no longer exists and thus the internal heat exchange necessary for high efficiency is no longer possible. The extension to this case is therefore necessary in order to understand and technically implement the design principles of electrolysis, which is essential for any “power-to-gas” technology.

The closed reversible cycle of an energy storage process based on H₂ described in FIG. 1 does not describe the more detailed design of the H₂O tank (6), the heat recovery via the condenser (5), the heat accumulators (7) and (8), and the evaporator (9), but only their ideal functioning. Thus, the system boundary within which these functions must be fulfilled is also freely selectable. It must only be possible to deliver H₂O, H₂ and O₂ reversibly to the respective reservoirs and to take them out again reversibly, as well as to exchange the reversible heats T·Δ^{R}S and T·Δsᵥ reversibly between fuel cell and electrolyzer. Assuming the possibility of reversible transport of H₂ from the electrolyzer to the fuel cell via, say, ideal conduits, the environment can serve as a reservoir of H₂O, and O₂. Then only the securing of the necessary reversible heat exchange between fuel cell and electrolyzer remains to be solved in order to be able to describe a reversible process control with the environment as storage.

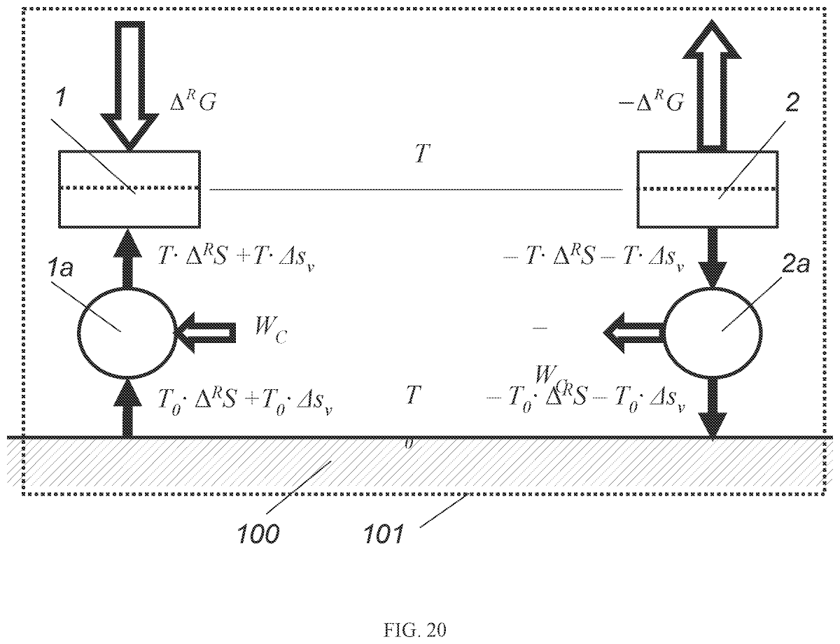

According to (Wolfgang Georg Winkler: Sustainable product development based on second law of thermodynamics. September 2011. Applied Energy 88(9): 3248-3256 DOI: 10.1016/j.apenergy.2011.03. 020), such a process control can be represented by a combination of reversible heat engine and reversible heat pump, as shown in FIG. 20. Between the temperature levels T and T₀, the heat (T₀·Δ^{R}S+T₀·Δsᵥ) coming from the fuel cell (2) (negative sign) is thus supplied to the environment (100) and from there to the electrolyzer (1) (positive sign) with the aid of Carnot processes, once operating as a heat engine (2a) and once as a heat pump (1a). For this purpose, the work WC must be dissipated once (negative sign) and supplied once (positive sign). The same principle is also applied to the outgoing and incoming substance flows from the fuel cell and electrolyzer (Wolfgang Georg Winkler: Sustainable product development based on second law of thermodynamics. September 2011. Applied Energy 88(9): 3248-3256 DOI: 10.1016/j.apenergy.2011.03.020), where the reversible work input or output corresponds to the associated exergy change of the respective substance flow. This overall system, which is independent of the distance between fuel cell and electrolyzer, is formed with the help of the system boundary (101), within which only reversible processes take place. The environment thereby forms a reversible storage for all heat and substances exchanged with it, while at the same time a reversible (electrical) energy network is tacitly assumed to be a reversible storage of the reversible work supplied and discharged. Furthermore, the choice of the environment as the storage eliminates the need to include the refilling of the storage in the considerations, because the reversible heat and substance extraction by the electrolysis system does not change the thermodynamic state of the environment. Similarly, the withdrawal of the required reversible Carnot work for the heat pump does not affect the state of a global reversible power supply system of ambient character. Thus, obviously, the electrolysis system for electricity storage is the most thermodynamically advantageous, which draws the required heat input from the environment with the least possible effort, converting the largest possible amount of electric work into thermodynamic potential of the formed H₂. In contrast, high-temperature electrolysis may be the more advantageous solution when high-temperature heat is sufficiently available and electric power is scarce or too expensive. Whereby it must not be neglected that high temperature heat can be used for electricity generation as is well known and thus its thermodynamic value is significantly higher than that of low temperature waste heat.

This also indicates the design principle of how, in principle, the losses to provide the required evaporation heat can be minimized in any isolated synthetic gas generation with H₂ as reactant. With the aid of a heat pump, the required evaporation evaporation heat can always be provided in a more energy-efficient manner in low-temperature electrolyzers than is the case with the dissipation of electrical work that is common today. The energy requirement of the heat pump can be further reduced if any waste heat can be used as its heat source instead of ambient heat. The heat pump can be dispensed with if the temperature of the waste heat is above the required evaporation temperature, usually 100° C. In the sense of the reference process, waste heat can be interpreted as any heat which, if not used for evaporation, would dissipate in the environment and thus contribute to its entropy increase.

The second main influence follows from the substance separation in thermal processes for CH₄ generation according to Sabatier. Here, too, all possible process paths are analyzed accordingly (Wolfgang Georg Winkler: Sustainable product development based on second law of thermodynamics. September 2011. Applied Energy 88(9): 3248-3256 DOI: 10.1016/j.apenergy.2011.03.020). A reversible CH₄ generation in principle would be the reversal of CH₄ oxidation according to:

2H₂O+CO₂=CH₄+2O₂ (5)

In contrast to H₂ production by electrolysis, there are currently no operational processes besides the thermal Sabatier process for CH₄ production to electrochemically remove O₂ from the reactor via an electrolyte. However, since O²⁻-conducting electrolyte materials are available as essential components for such processes and are constantly being further developed, and since catalyst development is also currently making interesting progress, the results of a corresponding thermodynamic analysis are included here. However, partial O₂ removal for product separation is currently only possible via upstream electrochemical H₂ or CO generation. In the first case, the reaction equation Eq. (5) must then be replaced by:

4H₂+CO₂=CH₄+2H₂O, (6)

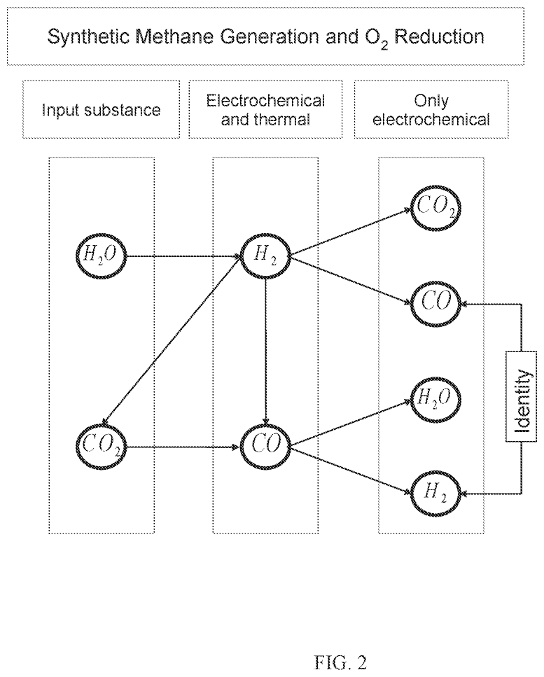

so that the reaction products can be separated by condensation of the H₂O. The disadvantage here is that the number of moles of H₂O supplied to the system (electrolyzer and thermal methanation reactor), and thus the required evaporation heat, must be doubled compared to Eq. (5). Therefore, the processes used to remove O₂ from the methanation reactor and thus in the preceding process steps have a decisive influence on the losses. A schematic diagram as shown in FIG. 2 serves to analyze the possible process steps. There, the starting materials and the thermodynamically possible variants of reactions are shown in 3 columns, with arrows indicating the corresponding links. H₂O and CO₂ enter the system as starting materials. The first step before thermal methanation is upstream electrolysis. For methanation, reduction to at least 2 mol of H₂ per mol of CH₄ to be produced is mandatory. To implement Eq. (6), H₂ generation is sufficient as the only electrochemical process because CO₂ is a reactant in Eq. (6). However, the reduction of CO₂ to CO can also be represented electrochemically and has the advantage that the reaction proceeds at ambient conditions without phase change. Thus, in addition to the methanation reaction Eq. (6), which requires the provision of 4 mol of H₂ per mol of CH₄, the thermal methanation reaction Eq. (7) is also an option, requiring only 3 mol of H₂ per mol of CH₄. This reduces the evaporation loss by 25% compared to the reaction Eq. (6). However, two different electrolysis processes are then required simultaneously:

3H₂+CO=CH₄+H₂O (7)

In the third column of FIG. 2, combinations are given only for those processes which are possible with an O²⁻-conducting electrolyte via which O₂ can be discharged electrochemically. The combination of 2 upstream electrolysis processes for the production of CO and H₂ fulfills this requirement, combined with the removal of ½ mol O₂ in the methanation reactor according to the reaction Eq. (8):

$$2 {H}_{2} + CO = {CH}_{4} + \frac{1}{2} {O}_{2} . \qquad ( 8 )$$

In an adaptation of Eq. (8), if electrolysis is omitted to produce CO, the reaction equation (9) follows, in which CO₂ is fed directly into the methanation reactor, in which 1 mol of O₂ must then be removed:

2H₂+CO₂=CH₄+O₂ (9)

Accordingly, it is also possible to adapt Eq. (7) in such a way that H₂ production is omitted and 2 H₂O is fed in. The electrolysis then produces only CO and in the methanation reactor 3/2 mol O₂ must then be removed according to Eq. (10):

$$2 {H}_{2} O + CO = {CH}_{4} + \frac{3}{2} {O}_{2} . \qquad ( 10 )$$

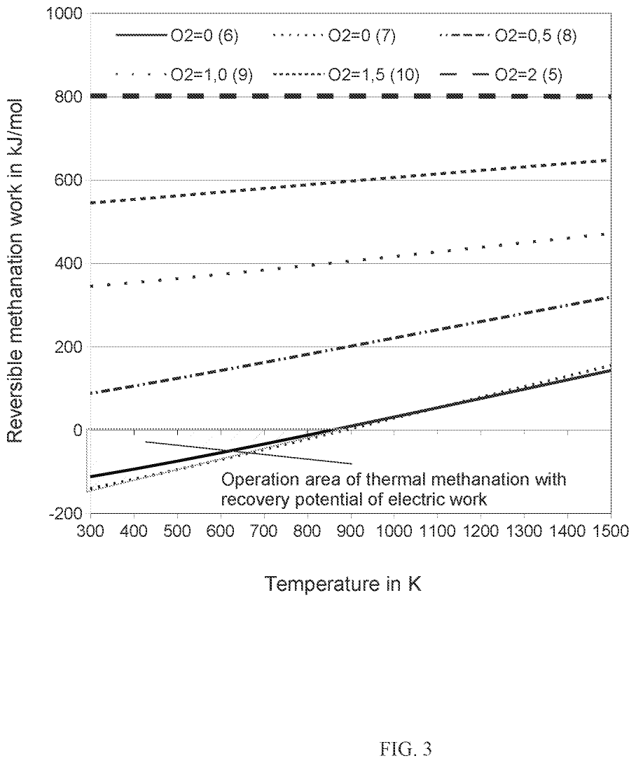

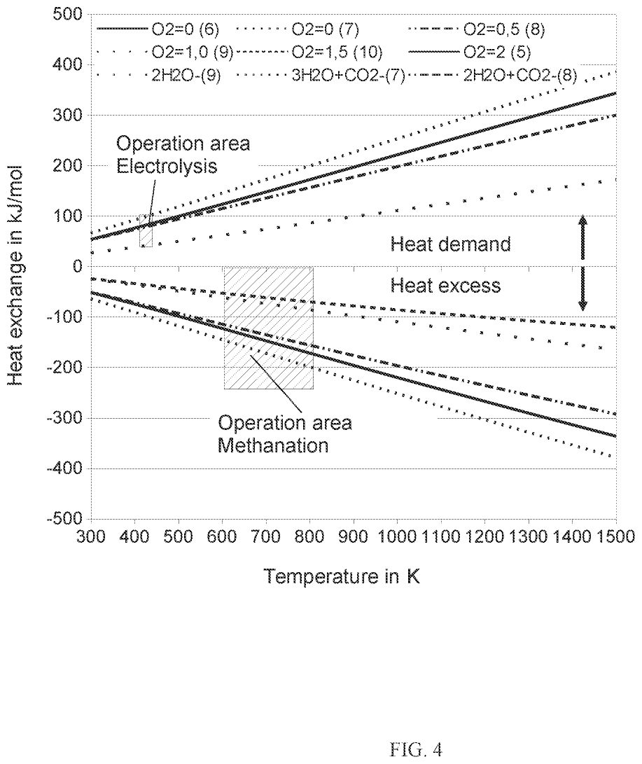

For the energetic evaluation it is still important, which reversible work is required for the reactions according to Eqs. (6) to (10), or what work, if any, they could deliver. This can be determined with the help of FIG. 3. There, above the reaction temperature, the reversible methanation work of the methanation reactions dealt with here, eqs. (5) to (10), with the amount of O₂ discharged as a parameter and with reference to the associated reaction equation. It becomes clear that the O₂ removal in the methanation reactor is very well suited together with the reactor temperature to describe the energetic reactor state. With the exception of a narrow operating range in which thermal reactions are possible, in the range of reactions described by Eqs. (5) to (10), work must always be supplied. The thermal reaction Eq. (6) is reversible only at 862 K and reaction Eq. (7) only at 892 K, where in each case the reversible work vanishes (Δ^{R}G=0). At lower temperatures, Δ^{R}G<0 holds and the methanation reactor could supply work, but due to the existing thermal reactor design, this work must be dissipated and removed as heat. At higher temperatures, work would have to be supplied to the methanation reactor because of Δ^{R}G>0. The conversion of the thermally driven processes according to Eqs. (6) and (7) into electrochemical processes with H⁺-conducting electrolytes, however, allows in principle to recover electric work during the methanation reaction. All of the above reversible methanation reactions Eqs. (6) to (10) always give off heat to cover exactly the demand of the upstream electrolysers.

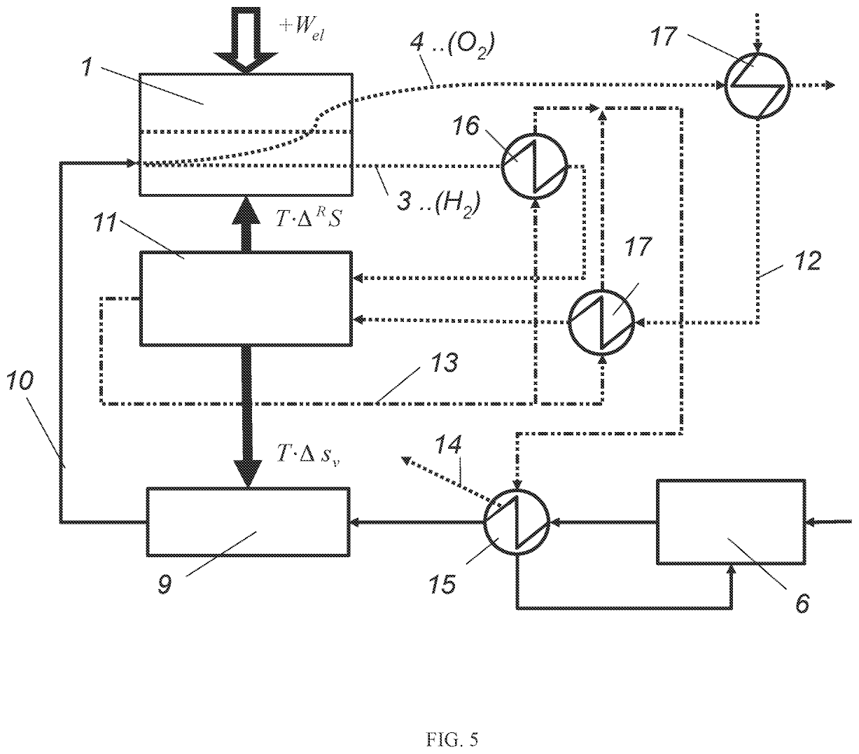

The combination of H₂O or CO₂ electrolysis with the reactions described by Eqs. (6) to (10) also requires a more detailed analysis of the heat fluxes in the linked reactions. Analogous to FIG. 3, FIG. 4 plots the heat release (−) from the methanation step and the total heat absorption (+) from the electrolyzers versus temperature. As the temperature increases, the amount of heat given off and the amount of heat absorbed increases. This indicates that therefore lower temperatures in the processes lead to lower investment costs. The working ranges of the electrolysis and methanation processes are also plotted, as is the amount of O₂ discharged as a parameter, and the assignment to the electrolysis and methanation processes can be seen from the equation numbers noted. Since the temperature of the methanation is usually higher than that of the associated electrolysis, more heat is given off than is needed for the electrolysis. This results in a surplus in heat output that can be used to evaporate the water for electrolysis. This effect occurs because electrolysis operating at a lower temperature requires more electrical power than at a higher temperature, where more energy is supplied by heat. These different temperatures are process-related because of the operating temperature of the catalysts, and the excess (irreversible) heat thus supplied can be used as evaporation heat in the system.

All processes described herein for methanation according to Eqs. (6) to (10) have three different temperature levels as a common feature of the process control. These are, with increasing temperature, the ambient state, the common temperature level of electrolyzer and evaporator, and the temperature level of the methanation stage. As discussed in (Wolfgang Georg Winkler: Sustainable product development based on second law of thermodynamics. September 2011. Applied Energy 88(9): 3248-3256 DOI: 10.1016/j.apenergy.2011.03.020), the ideal process can be approximated very well by a consistent heat exchange between the heating and cooling substance flows between the temperature levels. FIG. 5 serves to explain these circuits using the example of process control according to Eq. (6). Here, an O²⁻-conducting electrolyte in the electrolyzer (1) is taken as a basis and, for clarification, reference is made to the resulting assignment to O₂ and H₂. The methanation reactor (11) is supplied with H₂ via line (3) and with CO₂ via line (12). Of the heat released there, to cover the heat balance, the T·Δ^{R}S portion is supplied to the electrolyzer (1) and the T·Δsᵥ portion to the evaporator (9). The CH₄—H₂O mixture is discharged from the methanation reactor (11) via the line (13) according to Eq. (6). The CH₄—H₂O mixture is then passed through the heat exchangers (16) to the H₂ preheater and the second stage CO₂ preheater (17) finally to the condenser (15) for separation of CH₄ and H₂O. The condensed H₂O is stored in the vessel (6) and the CH₄ is discharged separately (14). To produce 1 mol of CH₄, 4 mol of H₂O must be supplied to the electrolyzer, half of which is thus provided by recirculation and the other from outside. The required H₂O is supplied to the evaporator (9) via the condenser (15) after preheating and to the electrolyzer (1) as steam via the line (10). The O₂ leaving the electrolyzer is fed via the line (4) to the heat exchanger (17) as the first preheating stage for preheating the incoming CO₂ and is cooled there.

With this preliminary work, the tasks set for improving the efficiencies of the synthetic production of H₂ and CH₄ and other hydrocarbons, generally hydrocarbon compounds, which behave according to the characteristic diagrams of FIGS. 3 and 4, and for their technical implementation can be specified in the following points. Depending on the availability of suitable catalysts and electrodes, the use of O²⁻-conducting electrolytes can also enable direct methanation according to Eq. (5). This means that for each mol of CH₄ produced, at least 2 mol of H₂O must be supplied. Naturally, the corresponding evaporation heat must be supplied to the system at the same time. Although the reaction entropy is vanishingly small for reactions according to (5), the reversible electrochemical methanation reactor in the comparative process must again be supplied reversibly from the outside with the evaporation heat (T₀·ΔR^{S+T}₀·Δsᵥ) according to FIG. 20.

### TASK OF THE INVENTION

1. to use the principles of the reversible comparison process according to FIG. 1 to improve the energy efficiency of “power-to-gas” technologies based on H₂ and synthetic hydrocarbons, generally hydrocarbon compounds, as elements of large-scale electricity storage and to use CO₂ as a raw material in closed cycles without releasing CO₂ to the environment;
2. improving the energy efficiency of H₂ generation using electrolysis through devices to provide H₂O in gaseous state upstream of the electrochemical electrolysis cell, largely independent of the operating temperature;
3. improvement of thermal integration of upstream electrolysis for H₂ generation and Sabatier exothermic methanation reaction, and utilization of the working potential of this reaction for recovery, thus improving efficiency.
4. improvement of thermal integration of upstream electrolysis for H₂ generation and exothermic methanation in potential new electrochemical processes.
5. system integration of the newly developed above gas generators into an electricity storage system and with integrated recycling for CO₂ as a sustainable industrial feedstock.
6. transfer of the process logic and device to other related process designs.

### Solution of the Task

The elaborated reversible process control of electricity storage with the aid of H₂, as indicated in FIG. 1, can be directly implemented with minor adaptations into a technically realizable device for electricity storage for grid stabilization with high efficiency, the design of which leads directly to the sought-after devices for energy-efficient generation of H₂ and advantageously CH₄ as well as other synthetic hydrocarbons, generally hydrocarbon compounds. Crucial for the transfer of the principles to a device is that the electrolyzer is always provided with the required H₂O only in the gas phase by a suitable heat recovery or use of waste heat, in order to avoid high heat losses due to the necessary H₂O evaporation.

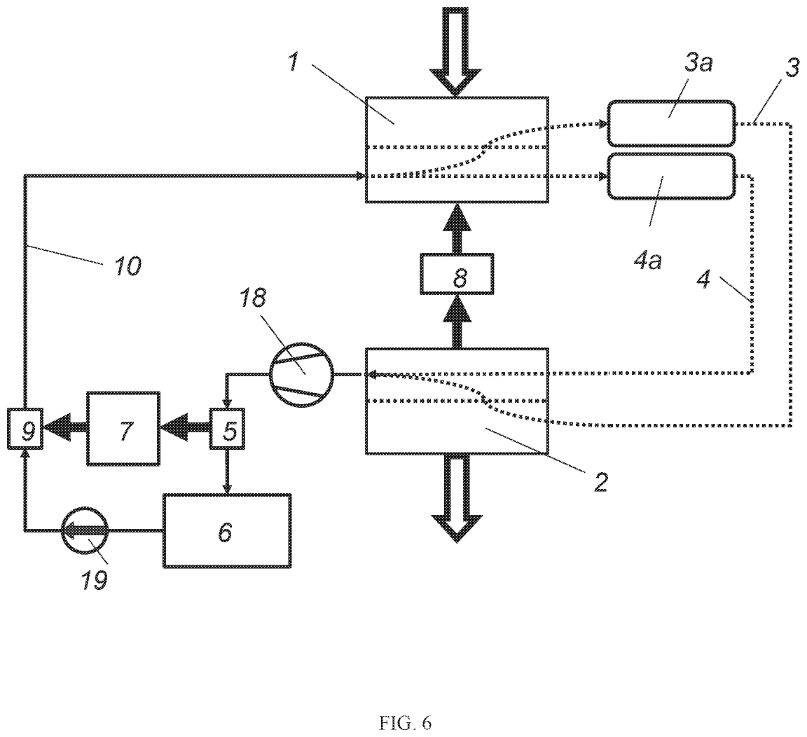

The technical solution of the electricity storage device, as shown in FIG. 6, differs from this reversible basic structure only in that real occurring temperature and pressure differences are taken into account. For this purpose, the fuel cell (2) is supplied with H₂ and O₂ from the gas accumulators (3a) and (4a) when electricity is required and is operated at a temperature so much higher than the electrolyzer (1) that reliable heat exchange via the heat accumulator (8) is ensured. The pressure of the exhaust steam of the fuel cell (2) is appropriately increased by a steam compressor (18) so that the heat accumulator (7) can be supplied by the condenser (5) with waste heat at a sufficiently high temperature during the condensation of the exhaust steam of the fuel cell, and the condensate is supplied to the H₂O tank (6). When there is a power surplus, the electrolyzer (1) is supplied with steam from the H₂O tank (6) via the feed pump (19), the evaporator (9) and the H₂O line (10), and then refills the two gas reservoirs (3) and (4) with H₂ and O₂. The heat required for this is taken from the heat accumulator (7).

The amount of heat stored in the heat accumulator (8), which follows from the release of the reaction entropy of the fuel cell, is relatively small at low operating temperatures of the electrolyzer. It is therefore advisable to check here whether the operating conditions of the electrolyzer permit an economical additional storage installation, or whether it is more sensible to compensate for the heat loss by electrical heating or to seek other solutions.

According to the invention, the evaporation heat from the heat source (22, 27) is waste heat from processes or waste heat obtained from the environment. Waste heat from (industrial) processes is in particular industrial waste heat, preferably with a temperature of maximum 400° C., further preferably maximum 300° C., still further preferably maximum 200° C. Waste heat from processes is advantageously external waste heat, i.e. it is supplied to the device according to the invention from outside and originates, for example, from an external industrial process and not from processes within the device according to the invention, in particular not from the waste heat of a fuel cell in the device according to the invention, unless this heat would otherwise be discharged into the environment as intended. A heat pump can be omitted if the temperature of the waste heat is above the required evaporation temperature.

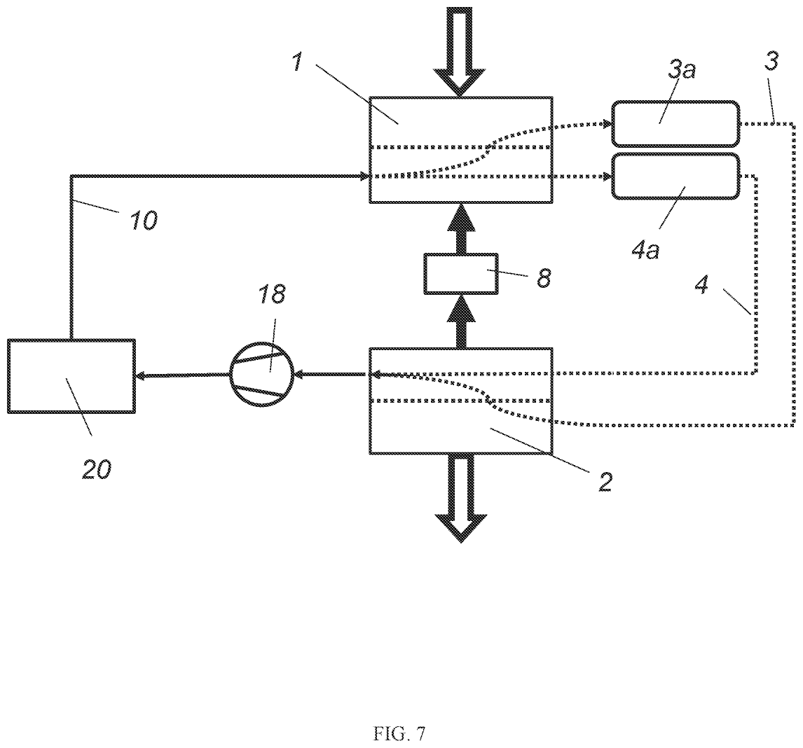

A simplified device shown in FIG. 7 with the same mode of operation differs from the basic solution discussed above in that the system of condenser (5), H₂O container (6), heat accumulator (7) and evaporator (9) is replaced by a steam accumulator (20) which supplies the electrolyzer (1) with steam in the event of a power surplus and is recharged by the exhaust steam from the fuel cell via the steam compressor (18) when power is required. To increase the security of supply, the steam accumulator (20) can be equipped with electrical heating for pressure maintenance or a connection to a steam network, if available, or further connected steam accumulators or other heat accumulators and thus be integrated into an industrial sector coupling with a large storage volume.

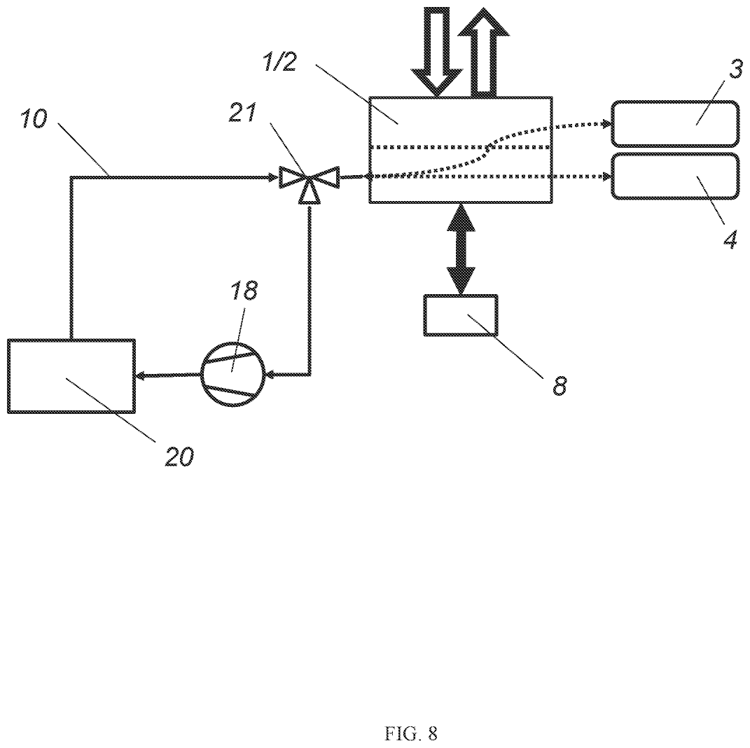

The device described can also be used with regenerative fuel cells (1/2), which can also be operated as electrolyzers, as FIG. 8 shows. For this purpose, the steam accumulator (20) is connected to the regenerative fuel cell (1/2) and the steam compressor (18) simultaneously via a three-way valve (21). In fuel cell mode, the three-way valve (21) connects the regenerative fuel cell (1/2) to the steam compressor (18) and the steam accumulator (20) is charged with the vaporous H₂O formed. In electrolysis mode, the three-way valve connects the steam accumulator (20) to the regenerative fuel cell (1/2) which is in electrolysis mode. To increase operational reliability and storage volume, the steam storage tank (20) can also be connected to other steam storage tanks in a steam network and integrated into an industrial sector coupling.

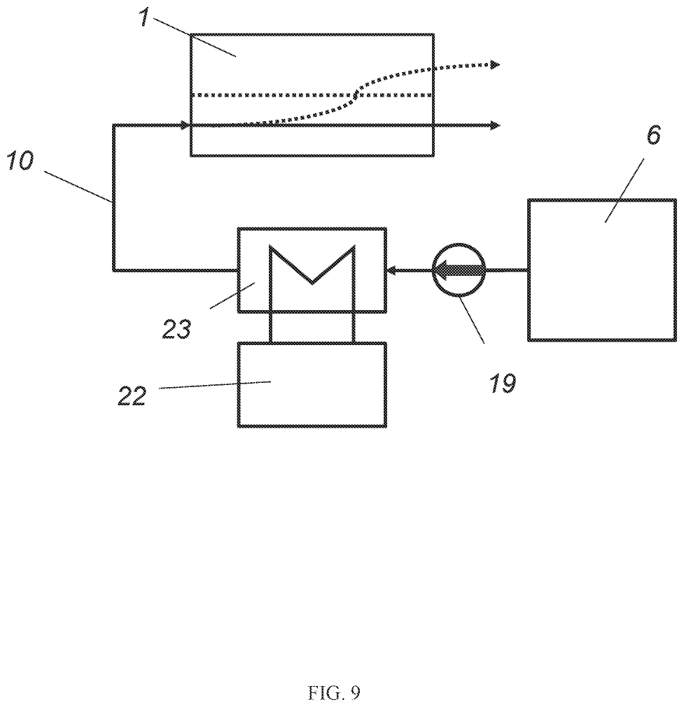

As already explained on the basis of the extension of the balance boundary of the electrolyzer-fuel cell system as shown in FIG. 20, the possibilities of process improvement shown in FIG. 1 can therefore also be used for open cycles for H₂ production and consequently for CH₄ production and, under certain conditions, also for the production of further hydrocarbons (CₙHₘ) or hydrocarbon compounds. Thermodynamically, the reversible process described above can be approximated if the evaporation heat released during H₂ oxidation—whether in a fuel cell or in a combustion reactor—is released to the environment during its condensation due to lack of recuperation capability, if conversely the evaporation heat of the H₂O required for electrolysis can be recovered from the environment. This can be achieved, or at least approximated, as part of a system integration of the electrolysis (1) using a circuit as indicated in FIG. 9. H₂O is fed from the tank (6) via the feed pump (19) to the evaporator (23), where it is supplied with sufficient heat of evaporation from the heat source (22), and the resulting steam is then fed to the electrolyzer (1). In its structural design, the evaporator (23) can integrate the steam accumulator (20) into the evaporator (9). If, for example, the heat source (22) is supplied with solar heat or geothermal heat with evaporation heat, the requirement for a reversible process would be well approximated, since the environment would have (at least approximately) resupplied the evaporator with the entropy given off during H₂ oxidation by this type of heat transfer. Although this ideal case is not always achievable, a setup according to FIG. 9 can always be used to utilize waste heat from various processes, if necessary within the limits of economic viability, with the aid of heat pumps, as already shown in the comparative process according to FIG. 20, to evaporate the H₂O supplied to the electrolyzer in order to significantly reduce the exergy losses of 17% during H₂ production. The advantage of this approach is that in industrial plants steam with low exergy can be used for this purpose in order to save the dissipation of electrical work.

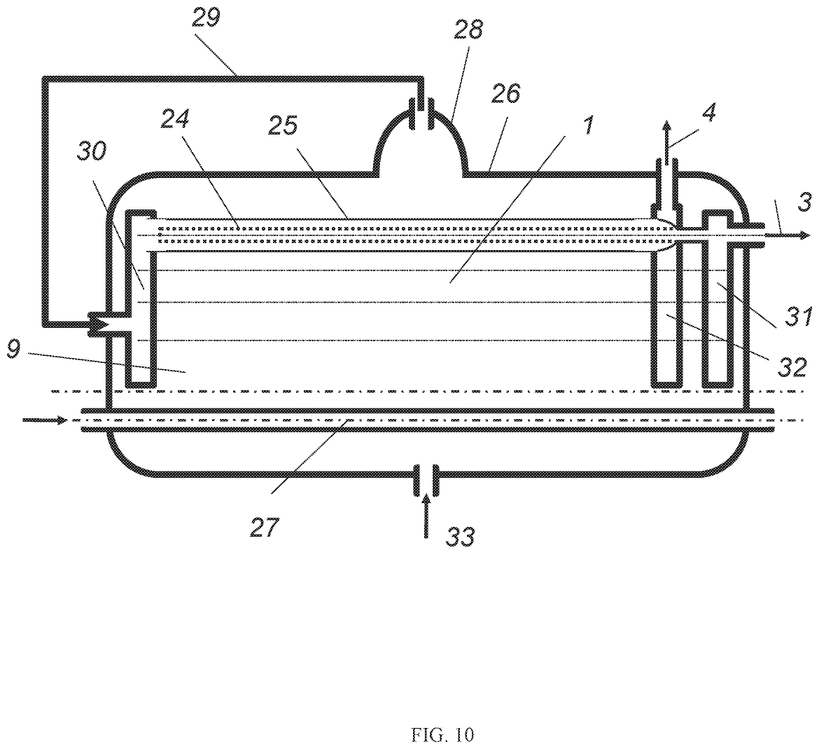

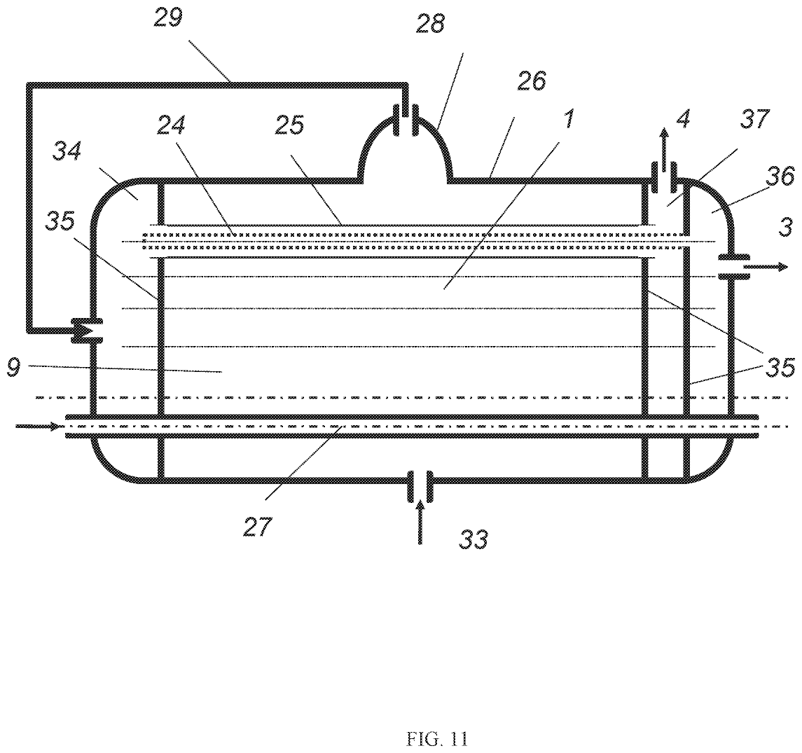

The device shown in FIG. 10 shows an advantageous installation of an electrolyzer (1) in an integrated evaporator (26), which also serves as a steam accumulator (20) or for steam storage, similar to a shell boiler. Heating can be carried out in various ways using a heat source (27). For example, various heat-emitting fluids can be passed through tubes, or exothermic reactions that need to be cooled can take place there. The heat sources (27) are to be arranged in parallel in such a way that an orderly evaporation process and water circulation in the evaporator section (9) can be ensured. The electrolyzer (1) consists of parallel arranged cell groups (24), which can be plate- or tube-shaped but also micro-process modules, which are surrounded by a cladding tube (25). The steam is directed via the steam dome (28), the steam line (29) to the distributors (30) and then to the cell groups (24) located in the cladding tube (25). The parallel flow distributors (30) connect the cell groups (24) and the associated cladding tubes (25) to the parallel headers (31) and (32). The gases of systems (3) and (4) then exit from headers (31) and (32). If an H⁺-conducting electrolyte is used for the cell groups (24), H₂ exits as a gas in (3) and O₂ exits as a gas in (4). When an O²⁻-conducting electrolyte is used, H₂ exits as a gas in (4) and O₂ exits as a gas in (3). H₂O is supplied via the inlet connection (33), whereby the entering water should already be preheated to close to the saturated steam temperature. When using the electrolyzer in industrial plants with steam networks, the inlet connection (33) can also be used to feed external steam to maintain the temperature of the system during shutdowns and also, depending on the possibilities of system integration, for steam heating instead of the heat source (27). For start-up and as an emergency supply, electrical heating of the evaporator is also possible. In the case of an integrated H₂ electricity storage system in accordance with the structures discussed in FIG. 7, the individual fuel cell groups (2) are surrounded analogously to the electrolysis cells (24) with or without cladding tubes corresponding to (25) and, while retaining the circuitry indicated in FIG. 7, are simultaneously used as a heat source (27). It should be noted that, in comparison with the design of the electrolyzer (1), the gases involved, H₂ and O₂, enter the fuel cell via two separate flow distributors in the fuel cell process in accordance with the process-related reversal of the flow direction, and the product H₂O is fed to the steam compressor (18) via an outlet collector and is then fed to the integrated evaporator (26) via the inlet (33). One option is for the steam compressor (18) to inject the higher pressure steam not only into the evaporator section (9), but also into the steam chamber (35a). Instead of electrical heating, gas accumulators (3a) and (4a) can also be used to supply heat to maintain the pressure of the integrated evaporator, and thus integrated H₂ burners supplied with O₂ that feed their exhaust gas H₂O directly into the evaporator section (9). In the case of integrated H₂ electricity storage systems corresponding to FIG. 8, the integration of the regenerative fuel cell (1/2) shown here in place of the electrolyzer (1) into the integrated evaporator (26) alone is sufficient, and the latter takes over the tasks of the steam accumulator (20) and the heat accumulator (8). The H₂O exiting the fuel cell (1/2) is returned to the integrated evaporator (26) via the steam compressor (18).

FIG. 11 shows a variation of the device shown in FIG. 10, in which the flow distributors (30) and the collectors (31) and (32) are replaced by chambers. The flow distributor (30) is replaced by the steam chamber (34), which is separated from the actual evaporator section (9) by a tube sheet (35). Similarly, the two headers (31) and (32) are replaced by the two gas chambers (36) and (37) formed with the tube sheets (35). The flow routing and gas distribution is analogous to what was said for FIG. 10. In the case of an integrated H₂ electricity storage unit according to the variants presented in FIG. 7 or 8, what has been said above in the description of FIG. 10 applies.

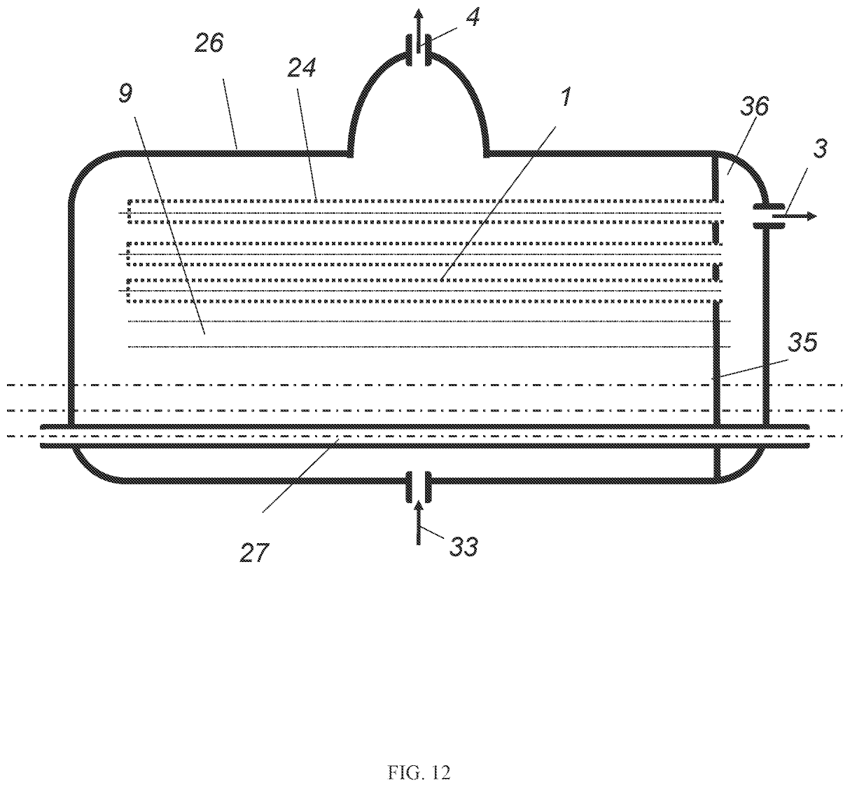

A further simplification of the construction of the device is shown in FIG. 12. The electrolyzer is constructed as in FIGS. 10 and 11 with the difference that the cladding tube (25) is omitted and the cells are arranged directly in the evaporator section (9). This reduces the number of tube sheets (35) to only one and the discharge of the generated gases O₂ and H₂ takes place via the associated outlet nozzles of lines (3) and (4). It is possible that steam is also entrained with the outgoing product gases O₂ or H₂. This effect can be largely reduced with cyclones and, if necessary, by subsequent condensation. Which gases escape at (3) and (4) depends on the electrolyte selected and corresponds to what has already been said above. In the case of an integrated H₂ electricity storage system according to the structures discussed in FIG. 7, the same applies as in the description of FIG. 10.

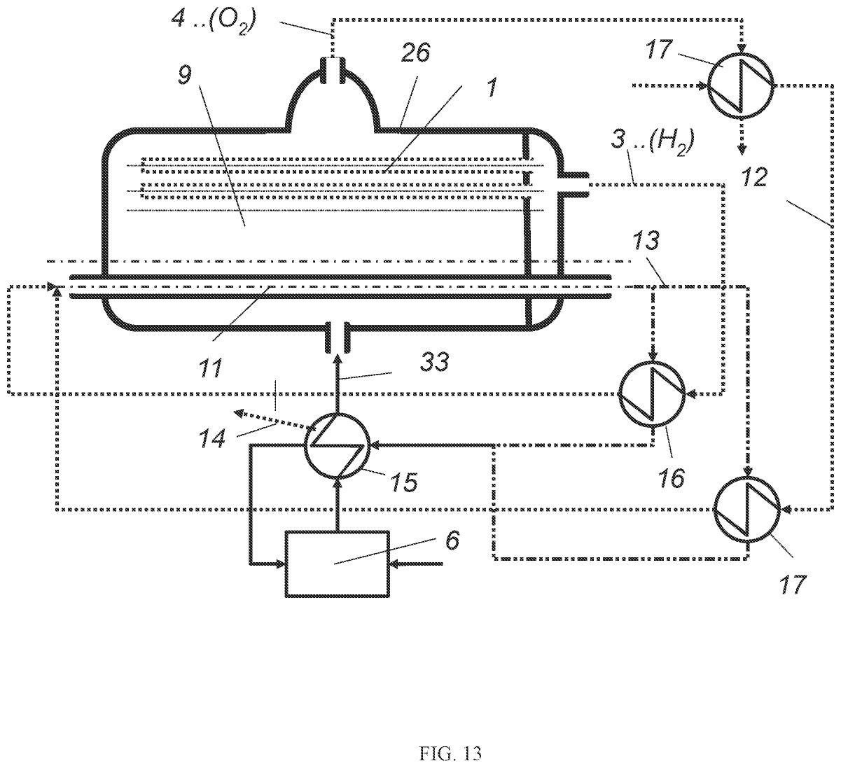

As FIGS. 3 and 4 show, the device shown in FIGS. 10 to 12 also fulfills very well the requirements for optimizing devices for CH₄ generation and its thermal integration with the upstream electrolysis. For this purpose, FIG. 13 shows the heating (11) of the integrated evaporator using the example of the variant shown in FIG. 12 by means of an exothermic reaction based on the methanation reaction Eq. (6) instead of or as a supplement to the general heat source (27). At the same time, this also shows the principle structure of the associated further necessary system integration for an integrated CH₄ generator. In principle, all of the systems integration steps given in Eqs. (6) to (10) are exothermic and therefore in principle suitable for heating the evaporator, as already explained for FIG. 4. The basic principles required to design integrated CH₄ generators combining electrolytic H₂ generation with both thermal and possible electrochemical CH₄ methanation reactors have already been presented in FIG. 5. For further clarification of the design principles of a highly integrated CH₄ generator, the reaction control according to Eq. (6) already selected and explained in FIG. 5 is therefore also further used in FIG. 13. As in FIG. 12, the assignment of system (4) to O₂ and of system (3) to H₂ is immediately clear here, because only H₂ and not O₂ is allowed to flow into the methanation reactor (11). The fluid in the piping system (12) is CO₂ and flows to the methanation reactor (11) without upstream electrolysis. The system design corresponds to the formulated rules described in (Wolfgang Georg Winkler: Sustainable product development based on second law of thermodynamics. September 2011. Applied Energy 88(9): 3248-3256 DOI: 10.1016/j.apenergy.2011.03. 020). According to these rules, reactants and products must first be used for heat recovery, taking into account the individual temperature levels of the reactions involved, before additional heat may be added. In the first stage, the products H₂ and O₂ are obtained from the reactant H₂O. In the second reaction stage, CH₄ and H₂O are produced from the reactants H₂ and CO₂ according to Eq. (6). This results in three different temperature levels: ambient, evaporator/electrolyzer and methanation, between which the heat recovery shown here takes place. The CH₄—H₂O mixture is discharged from the methanation reactor (11) via the line (13) according to equation (6). The CH₄—H₂O mixture is then passed through the heat exchangers (16) to the H₂ preheater and the second stage CO₂ preheater (17) and finally to the condenser (15) for separation of CH₄ and H₂O. The condensed H₂O is stored in the tank (6) and the CH₄ is discharged separately (14).

From the electrolyzer, 4 moles of H₂O are required to produce 1 mole of CH₄, half of which is thus provided by the recirculation and the other from outside. These 4 mol H₂O/mol CH₄ are supplied to the evaporator section (9) via the condenser (15) after preheating via the line (33) and are supplied internally to the electrolyzer (1) as steam. The O₂ leaving the electrolyzer is used via the heat exchanger (17) as the first preheating stage to preheat the incoming CO₂. In this process, the condenser (15) can also serve as a waste heat source for the heat pump.

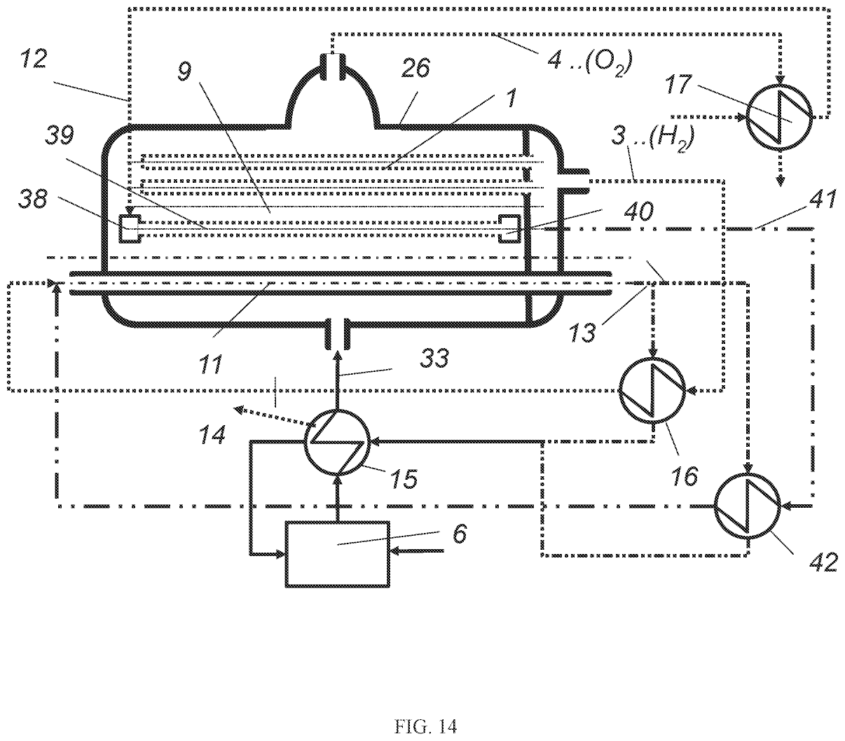

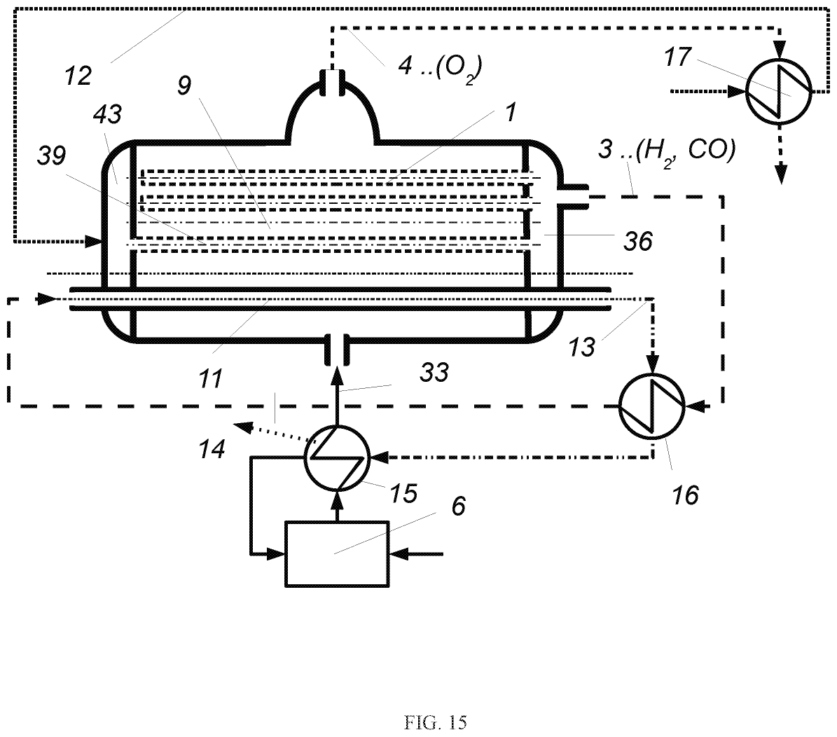

Since the electrolytic H₂ generation must also be combined together with the electrolytic generation of CO, FIG. 14 shows a corresponding addition to the system setup of the device shown in FIG. 13. However, the changes in the system only affect the area of the device that comprises the supply line of the CO₂ (12) and the components associated with it. In particular, these relate to the change in preheating associated with the supply and discharge of the process gases CO₂ and CO. The second preheating stage of CO₂ is replaced by a preheating of CO by the CH₄—H₂O mixture in the heat exchanger (42) corresponding to the temperature level. The design options for integrating the CO₂ electrolyzer (39) can be taken from FIGS. 10 and 11 using the example of integrating the H₂O electrolyzer. The solution shown in FIG. 14 is based on FIG. 10. When simultaneously integrating the electrolyzers for the generation of H₂ and CO, special care must be taken to avoid short circuits in the integrated evaporator (26). The CO₂ is fed via the line (12) to the distributor (38) of the CO₂ electrolyzer (39), where it is converted into CO while releasing ½ O₂ into the evaporator section (9) in the same way as for H₂O electrolysis. This is led via the collector (40) and the line (41) to the preheater (42) and from there to the methanation reactor (11). The conception of the treatment of the CH₄—H₂O mixture leaving the methanation reactor (11) remains unchanged compared to FIG. 13.

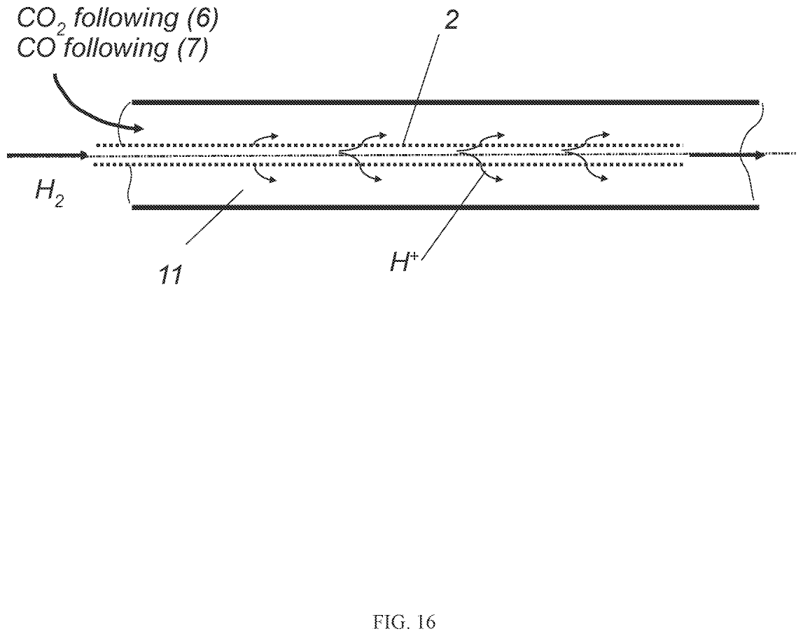

Analogous to FIG. 11, this device can be further simplified in terms of design, as shown in FIG. 15. There, the distributor (38) is replaced by a CO₂ inlet chamber (43), which serves to supply gas to the electrolysis cell (39). The CO formed in the electrolysis cell (39) is then fed into the H₂ outlet chamber (36) and the H₂—CO mixture is fed to the methanation reactor (11) via the line (3) after preheating in the heat exchanger (16), as in FIG. 13.

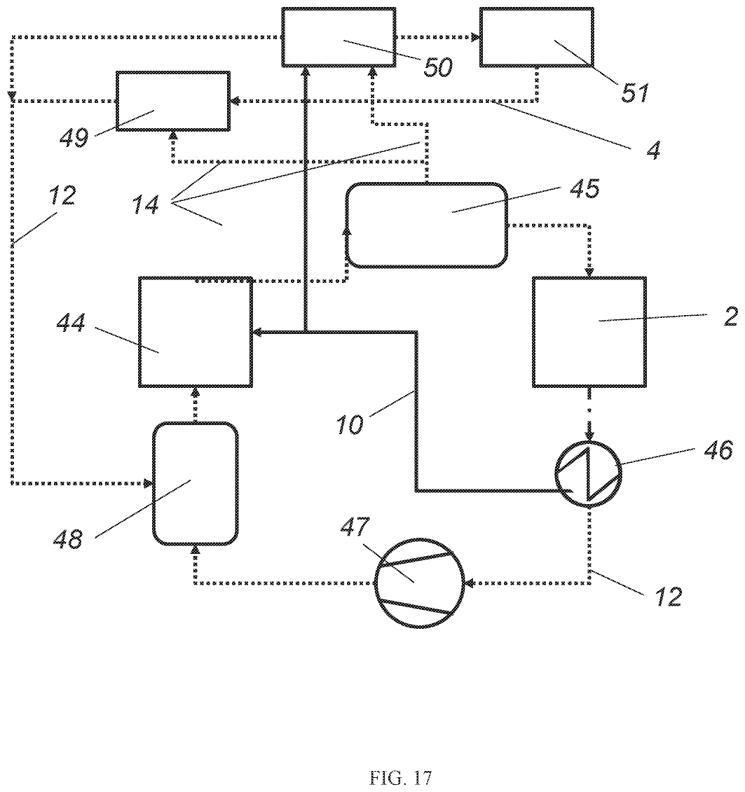

Another possibility for improving the efficiency of methanation according to Eqs. (6) and (7) results from utilizing the unused potential indicated in FIG. 3 for the recovery of electrical work in these thermal processes. FIG. 16 serves to explain the practical implementation. H₂ and CO₂ flow into the methanation reactor (11) according to Eq. (6) or CO according to Eq. (7). In both cases, additional H₂ alone is needed compared to the CH₄ generation requirement, in order to be able to separate O₂ after oxidation with H₂ by condensation of CH₄. The available potential to supply electric work can be utilized by adding H₂ using an H⁺-conducting fuel cell. H⁺ ions then emerge from the outer surface of the fuel cell and react with CO₂ and CO, respectively, to form CH₄ and H₂O. In all highly integrated systems according to FIGS. 10 to 16, special care must be taken to ensure that no short circuits can occur between the integrated current-carrying components and that they are excluded by design.

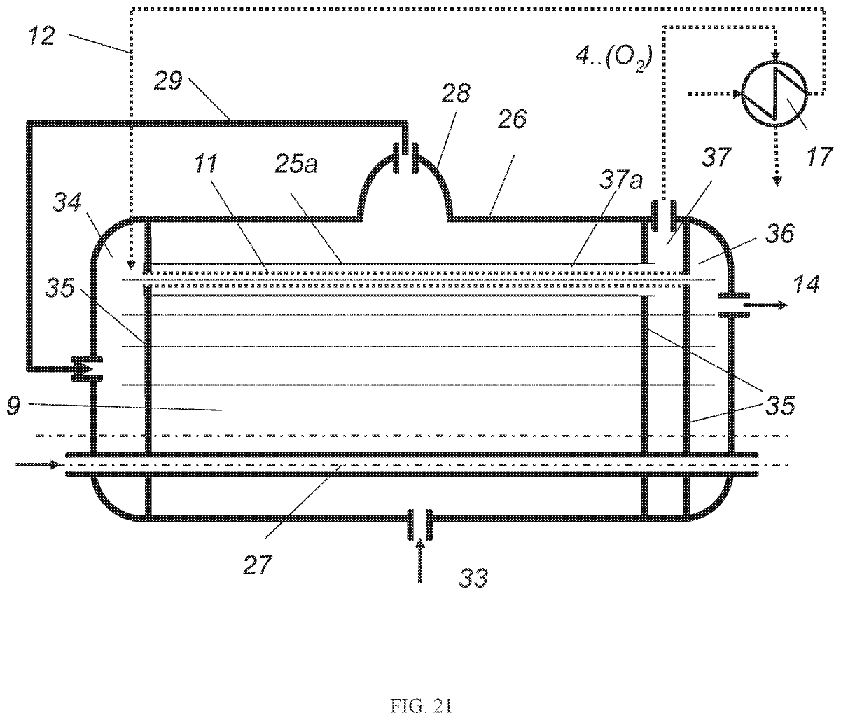

In the case of direct electrochemical generation of CH₄ using O²⁻-conducting electrolytes, already discussed above, the design principles derived above can be adapted to the appropriate devices. FIG. 21, which arises from the design according to FIG. 11, shows as an exemplary example a device for a methanation reaction according to Eq. (5). The electrolysis cells (24) are replaced by methanation reactors (11), the walls of which are formed from O²⁻-conducting electrolytes, whereby the channel (37a) is formed with the cladding tube (25a), which serves to discharge the O₂ produced during the reaction. Steam is supplied to the steam chamber (34) via the steam line (29). CO₂ is supplied there through the line (12) after its preheating in the heat exchanger (17), and the mixture of H₂O CO₂ is then fed into the methanation reactor (11). According to the design of the separate gas inlet by means of distributors or chambers according to FIG. 10 or FIG. 11, a separate feed of CO₂ and vaporous H₂O into the methanation reactor can also take place. Depending on the catalysts used, different operating temperatures are possible for such methanation reactors, therefore, due to the possible different operating conditions, reference is made to the already quoted design rules for heat recovery in (Wolfgang Georg Winkler: Sustainable product development based on second law of thermodynamics. September 2011. Applied Energy 88(9): 3248-3256 DOI: and to the presented embodiments in FIGS. 10 to 15.

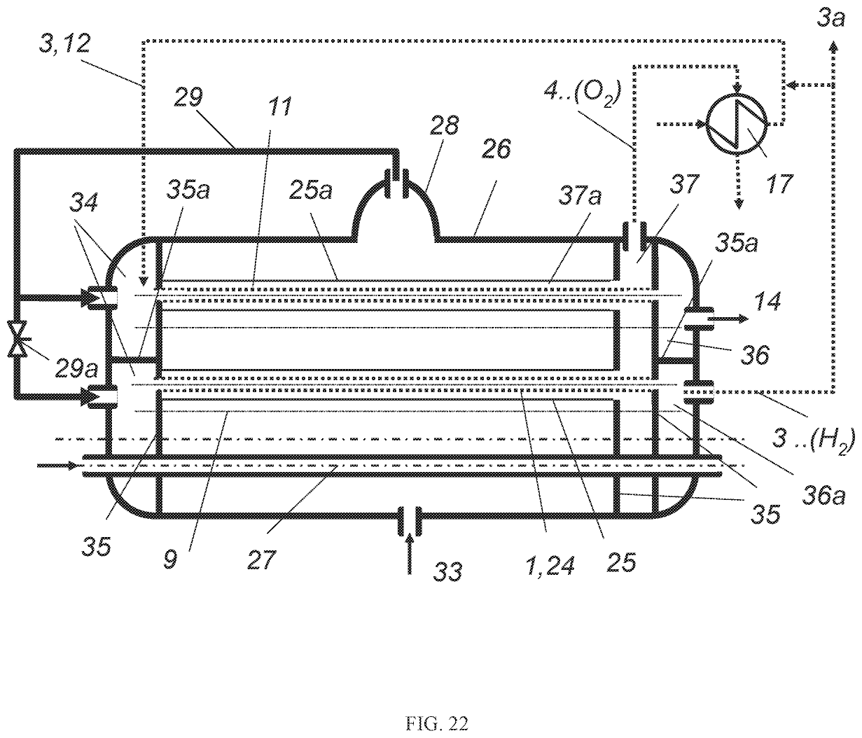

Using combinations with the devices for generating H₂ and CH₄ according to Eqs. (6) to (10), the inlet gas concentrations of the methanation reactors (11) can be optimized for different catalysts. For this purpose, the device according to FIG. 21 only has to be supplemented by the installation of H₂O and/or CO₂ electrolysis cells according to the embodiments of FIGS. 10 to 15. If, for example, an admixture of H₂ is desired, the device according to FIG. 21 can be combined with H₂O electrolysis, as FIG. 22 shows using the example of Eq. (9). For this purpose, only the chambers (34, 36, 37) at the inlet and outlet must be divided with a partition (35a) and/or supplemented or replaced with the design of flow distributors (30) and collectors (31), (32). The corresponding division at the outlet results in a chamber (36) for CH₄ and a chamber (36a) for H₂. The construction of the electrolysis section corresponds to FIG. 11 with an O²⁻-conducting electrolyte. The H₂ formed is admixed to the CO₂ line (12) via the line (3). Alternatively, the H₂ can also be added on the steam side in the line (29) or in the inlet chamber (34); in this case, an optional H₂ withdrawal is also possible via the branch (3a). A control device (29a) is used to control the amount of steam supplied to the electrolysis (1, 24) and thus the H₂ production. In the same way, the supply of CO and CO₂ to the methanation reactors (11) can be controlled in the required ratio. If only a smaller addition of H₂ is required for methanation, the cladding tube (25) can be dispensed with, following FIG. 12, if an H⁺-conducting electrolyte is used, the steam and the H₂ formed being fed to the methanation reactor (11) via the evaporator section (9) and the line (29). The O₂ exiting the electrolyzer (1, 24) is then discharged via the gas compartment (37), and the partition (35a) and gas compartment (36a) are omitted. The installation of a CO₂ electrolyzer (39) is analogous to that of an H₂O electrolyzer (1) with O²⁻-conducting electrolytes and accordingly a cladding tube analogous to (25) and with the associated gas chambers (43) and or flow distributors (38) and collectors (40). The pipe (41) conducts the formed CO to the methanation reactor (11). The geometrical arrangement of the individual integrated methanation reactors (11) and the electrolysers (1) and (39) is decisively determined by their influence on the heat and substance concentrations as well as the heat and substance transport and, if necessary, optimized with internals for flow control.

The further integration of the device of an integrated CH₄ generator (44) designed according to the above mentioned design principles into a sustainable energy system is essential for the sustainability and CO₂ freedom of its operation. Again, the reversible comparative process shown in FIG. 1 represents the theoretical basis of the process control. Accordingly, the CH₄ produced is stored in existing gas storage tanks (45) and used in fuel cells (2) to generate electricity and heat. The resulting flue gas contains only CO₂ and H₂O, and residual heat utilization in a flue gas condenser (46) allows CO₂ and H₂O to be separated. After compression in the CO₂ compressor (47), the resulting CO₂ is fed via a CO₂ piping system (12) to the CO₂ storage (48), which in turn feeds the CH₄ generator (44) again. To improve the sustainability of the energy system and the raw material economy, it is expedient to cover the CₙHₘ demand, generally demand for hydrocarbon compounds, of industry and commerce (49) from the gas storage facilities (45). However, since CH₄ synthesis requires CO₂ as a feedstock, this supply relationship of regenerative CH₄ supply inevitably results in the unavoidable need to also recycle CO₂ from plastic waste and return it to the CH₄ generator through the pipeline (12). Since the volumetric energy density of CH₄ is about four times higher than that of H₂, CH₄ can be used to hold significantly more energy to cover seasonal longer severe supply shortages of renewable power than would be possible with H₂. Therefore, it is appropriate to conceptually provide for the possibility of H₂ generation from CH₄ (50) to secure H₂ supply even in the event of prolonged generation shortfalls of renewable electricity generation, thus significantly increasing H₂ supply security. Conversely, hydrogen supply (4) to industry from ongoing hydrogen production (51) is also a useful addition. The H₂O system (10), which is also shown, is intended to illustrate the H₂O demand of the processes described, but in practice this is probably only of interest at highly integrated industrial sites.

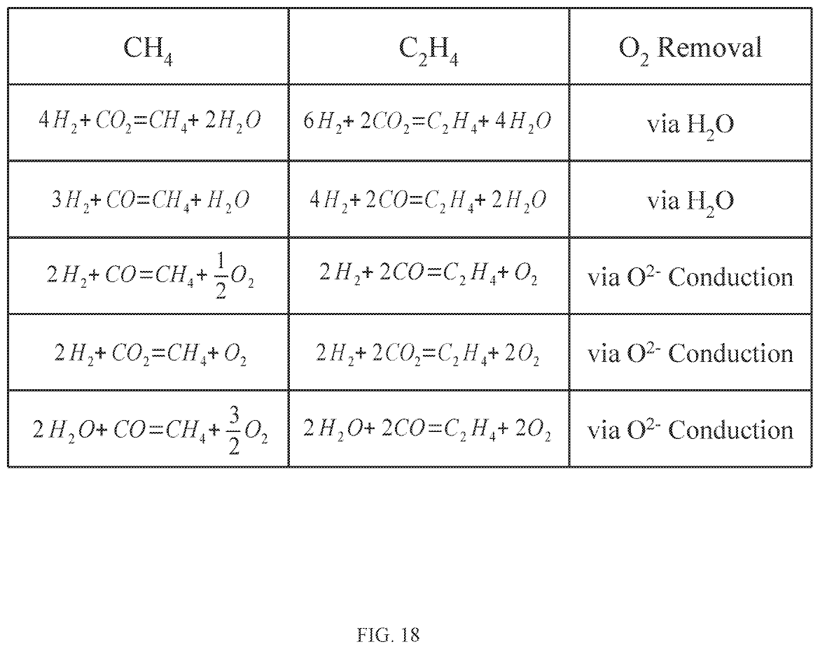

The process control of the devices derived here from the example of CH₄ production and the technical solutions shown here for their plant engineering implementation can also be used, as already indicated several times, for the production of C₂H₄ and other hydrocarbons CₙHₘ or hydrocarbon compounds. The prerequisite for this is that the thermodynamics of their process control correspond to the characteristic diagrams of reaction work, reaction heat and O₂ removal in the electrolyzers and in the methanation reactor shown in FIGS. 3 and 4, so that these structures can be used. FIG. 18 shows an example of a compilation of comparable reaction equations for CH₄ and C₂H₄ and the associated methods of O₂ removal.

1-17. (canceled)

18. A device, comprising:
an electrolyzer,
an H₂ gas reservoir for storage of H₂ gas,
an O₂ gas reservoir for storage of O₂ gas,
wherein the electrolyzer supplies the H₂ gas reservoir with H₂ gas produced via electrolysis and supplies the O₂ gas reservoir with O₂ gas produced via electrolysis,
an associated H₂ gas line,
an associated O₂ gas line,
an H₂O container, and
an evaporator,
wherein the electrolyzer is supplied with vaporous H₂O from the H₂O container via a feed pump, the evaporator, and a vaporous H₂O line,
wherein H₂O in the evaporator is supplied with evaporation heat from a heat source, wherein the evaporation heat from the heat source is waste heat from processes or waste heat obtained from the environment.

19. The device according to claim 18,
wherein the waste heat used for evaporation is heated by a heat pump to a temperature level required for evaporation.

20. The device according to claim 18, further comprising:
a fuel cell,
wherein the electrolyzer is arranged for supplying the fuel cell with H₂ gas via the associated H₂ gas line and O₂ gas via the associated O₂ gas line,
wherein a device for supplying waste heat of the fuel cell to a first heat accumulator for supplying heat to the electrolyzer, and for conducting the gaseous reaction product H₂O of the fuel cell via a vapor compressor and a condenser, which delivers waste heat to a second heat accumulator, to the H₂O container, and from there, if required, conducting the H₂O via the feed pump and the evaporator supplied by the heat accumulator via the line in the vapor state to the electrolyzer.

21. The device according to according to claim 18,
wherein the electrolyzer and the heat source are integrated in an integrated evaporator, which also serves as a steam accumulator, with electrolytic cells protected by cladding tubes and are supplied from a steam dome via a line and flow distributors or a steam space, which is separated from an evaporator section by perforated plates, with vaporous H₂O and the product H₂ gas and O₂ gas are discharged correspondingly either via collectors and or gas chambers, again separated by the perforated plates, via the lines, the H₂O supply being effected via one or more connections.

22. The device according to claim 21,
wherein the port or ports are also used for steam supply from a steam network.

23. The device according to claim 21,
wherein the heat supply of the heat source is provided by heat emitting fluids or reactions from outside.

24. The device according to claim 20,
wherein, in the case of an electricity storage device, fuel cells are used as heat sources.

25. The device according to claim 18,
wherein the electrolyzer and the heat source are integrated into an integrated evaporator which also serves as a steam storage,
wherein cells of the electrolyzer are arranged directly in an evaporator section without cladding tubes and the steam can flow directly to electrodes of the cells,
wherein, in the case of an H⁺-conducting electrolyte, an O₂ outlet is provided and H₂ is discharged via a gas space and an H₂ outlet,
wherein, in the case of an O²⁻-conducting electrolyte, the gases in the outlets are correspondingly interchanged and, accordingly, also the associated connecting lines.

26. The device according to claim 18, further comprising:
a methanation reactor for the additional production of methane CH₄ from H₂ and CO₂ or from H₂ and CO or from H₂O and CO, lines, heat exchangers, and
a CH₄ discharge in an H₂O condenser,
wherein the methanation reactor serves as a heat source for the electrolyzer.

27. The device according to claim 26,
wherein the device is arranged for conducting incoming CO₂ via a distribution system or a separated gas space to a CO₂ electrolysis cell for the generation of CO via electrolysis, and for feeding CO generated there via an outlet collector and the conduit after preheating or after mixing with H₂ in the gas space as synthesis gas to the methanation reactor.

28. The device according to claim 26,
wherein the H₂ supply for methanation is via an integrated fuel cell with an H⁺-conducting electrolyte.

29. The device according to claim 26,
wherein the device for storing CH₄ generated in a CH₄ generator in gas storage tanks for use in fuel cells for power and heat generation and for feeding the formed CO₂ via a flue gas condenser and a CO₂ conduction system after compression in a CO₂ compressor to the CO₂ storage tank and from there to the CH₄ generator for renewed CH₄ generation with H₂O, further arranged for the production of H₂ and CO₂ from CH₄ via reforming reactors and supply of the produced CO₂ via the line to the CO₂ storage.

30. The device for producing CH₄ and H₂ according to claim 26,
wherein the device is arranged for producing ethene C₂H₄ from H₂ and CO₂ or from H₂O and CO, and/or other hydrocarbons CₙHₘ.

31. The device according to claim 26,
wherein the methanation reactor is provided with an O²⁻-conducting electrolyte which allows O₂ forming during methanation to be removed in situ during the reaction.

32. The device according to claim 26,
wherein the methanization reactor is installed in the integrated evaporator, the walls of which are formed of O²⁻-conducting electrolytes, whereby the channel is formed with the cladding tube, which channel serves for the discharge of the O₂ produced during the reaction,
wherein the methanation reactor is supplied with vaporous H₂O are via a steam dome via a conduit and a steam compartment, which is separated from an evaporator section by perforated plates, or via flow distributors, and
wherein the product gases CH₄ and O₂ are discharged correspondingly either via headers or gas compartments, again separated by the perforated plates, via the lines,
wherein the H₂O supply and/or steam supply from a steam network is effected via one or more connections,
wherein the heat supply is effected by any heat-emitting fluids or reactions from outside.

33. The device according to claim 26,
wherein the methanation reactor is additionally supplied with H₂ and/or CO via electrolyzers integrated in the integrated evaporator,
wherein H₂ and/or CO, when an O²⁻-conducting electrolyte is used, is supplied to the gas compartment via the gas compartment and the line and, when separate H₂ is supplied to the gas compartment, via the gas compartment and the line with the extraction point, and, in the case of separate CO routing, via collectors and the line to the gas compartment, whereby, when an H⁺-conducting electrolyte is used, H₂ can be routed separately via the evaporator section, the steam line and the gas compartment, the released O₂ being removed via the gas compartment and CO being routed via collectors and the line to the gas compartment.

34. The device according to claim 26,
wherein the heat released at larger temperature differences between methanation and electrolysis is used to evaporate the supplied water.

35. The device according to claim 18,
wherein the evaporator can be kept ready for operation even in the event of failure of the heat supply by an external heat supply by electrical heating, direct H₂/O₂ combustion in the steam compartment, or external steam supply.

36. The device according to claim 18,
wherein a gas outlet from the electrolysis located directly downstream of the evaporator is provided with separating devices, such as cyclones or condensers, in order to avoid steam outlets with the electrolysis gas.

37. The device according to claim 18,
wherein the condenser also serves as a waste heat source of the heat pump.
